(19) United States
(12) Patent Application Publication
(10) Pub. No.: US 2015/0045251 A1
(43) Pub. Date: Feb. 12, 2015

(54) DUPLEX CHROMOGENIC ASSAY FOR IN SITU DETECTION OF NUCLEIC ACIDS

(71) Applicant: Advanced Cellular Diagnostics, Inc., Hayward, CA (US)

(72) Inventors: Li-Chong Wang, Foster City, CA (US); Nan Su, San Ramon, CA (US); Xiao-Jun Ma, Pleasanton, CA (US); Yuling Luo, San Ramon, CA (US)

(21) Appl. No.: 14/381,942

(22) PCT Filed: Mar. 6, 2013
(86) PCT No.: PCT/US13/29467

§ 371 Date: Aug. 28, 2014

Related U.S. Application Data
(60) Provisional application No. 61/607,344, filed on Mar. 6, 2012.

(51) Int. Cl. C12Q 1/68

(52) U.S. Cl. CPC C12Q 1/6841; C12Q 2600/16
USPC 506/9

(57) ABSTRACT

The disclosure provides methods of detecting two or more target nucleic acids. The methods can include the steps of contacting a sample with two or more label probes having distinct enzyme labels and targeting distinct nucleic acid targets, binding the two or more label probes to the target nucleic acids by hybridization; contacting the sample with a first substrate for the first enzyme of the first label probe; reacting the first substrate with the first enzyme, thereby producing a first detectable signal; contacting the sample with a second substrate for the second enzyme of the second label probe; reacting the second substrate with the second enzyme, thereby producing a second detectable signal; and detecting the first detectable signal and the second detectable signal, thereby detecting the first and second target nucleic acids in the sample.

This application claims the benefit of priority of U.S. Provisional application Ser. No. 61/607,344, filed Mar. 6, 2012, the entire contents of which are incorporated herein by reference.

### BACKGROUND OF THE INVENTION

The present invention relates generally to nucleic acid detection and more specifically to in situ detection of nucleic acids.

In situ detection of RNA is very important in life science research and diagnostics because it allows the molecular signals to be mapped to relevant cells, which can provide additional insights into mechanisms of the biological system under study. In order to detect or visualize RNA, reporter molecules are used that attach to the target RNA molecule and produce detectable signal. Depending on the type of reporter used, the most popular detection methods can be categorized into fluorescent and chromogenic detection. In fluorescent detection, the reporter is a fluorophore, which emits distinctive fluorescent light under optical excitation. In chromogenic detection, the reporter is generally an enzyme that reacts with a substrate to produce a colored deposit at the reporter site.

Compared with fluorescent detection, chromogenic detection can be visualized directly using a common bright field microscope, which is convenient and makes it easier to observe and analyze cell morphology and tissue context. However, it is generally believed that it is easier to detect multiple target RNAs using fluorescent detection methods.

Most existing multiplexed chromogenic detection methods use a sequential chromogenic reaction approach, which involves attaching one enzyme reporter to one target followed by reacting with an associated chromogenic substrate to produce a color deposit; then the second enzyme reporter is attached to the second target followed by reacting with a second substrate to produce a different color deposit, and so on. Such a sequential approach has the obvious disadvantage of a prolonged, complex assay procedure. In addition, the insoluble color precipitates produced in the preceding chromogenic reaction can prevent the subsequent reporter(s) from attaching to their respective target(s) in later steps. Furthermore, sequential approaches using the same enzyme on both reporters are very prone to signal leak-through. If the first enzyme is not completely inactivated, the continued reaction with the second substrate will lead to a false positive signal for the second target.

There have been reports of a simultaneous labeling approach used in immunohistochemistry (IHC), in which multiple different enzymatic reporters are attached to different target molecules in a single assay step. These labeled enzyme reporters then react with their corresponding different substrates sequentially to produce deposits of different colors. Such an approach is not commonly adopted because the activity of a particular enzyme is highly dependent on its unique buffer condition. The enzyme-substrate reaction has to be done individually. The activity of the enzyme(s) in later steps can be damaged by the enzyme-substrate reaction in the previous steps, resulting in a much weakened signal for targets detected in later steps.

Detecting multiple target nucleic acid molecules in the same sample in a single assay is important because it helps to reveal the biological relationship between different molecular biomarkers. Thus, there exists a need for assays that allow detection of multiple target nucleic acid molecules in a sample in a single assay. The present invention satisfies this need an provides related advantages as well.

### SUMMARY OF INVENTION

The invention provides methods of detecting two or more target nucleic acids. The methods can include the steps of comprising contacting a sample with two or more label probes, wherein each label probe comprises a distinct enzyme label and targets a distinct nucleic acid target; binding the two or more label probes to a first target nucleic acid and a second target nucleic acid in the sample by hybridization; contacting the sample with a first substrate for the first enzyme of the first label probe; reacting the first substrate with the first enzyme, thereby producing a first detectable signal associated with the first target nucleic acid molecule; contacting the sample with a second substrate for the second enzyme of the second label probe; reacting the second substrate with the second enzyme, thereby producing a second detectable signal associated with the second target nucleic acid molecule; and detecting the first detectable signal and the second detectable signal, thereby detecting the first and second target nucleic acids in the sample.

The invention additionally provides a method of detecting two or more target nucleic acids. The methods can include the step of contacting a sample with two or more sets of label probes, wherein each set of label probes comprises a plurality of label probes, wherein the plurality of label probes within a set have the same enzyme label, and optionally wherein the plurality of label probes comprise label probes that attach or bind to different regions of a target nucleic acid molecule; and wherein each set of label probes comprises a distinct enzyme label and targets a distinct nucleic acid target; attaching or binding the two or more sets of label probes to two or more target nucleic acids, for example, at least a first target nucleic acid and a second target nucleic acid, or additional target nucleic acids, in the sample by hybridization; contacting the sample with a first substrate for the first enzyme of the first set of label probes; reacting the first substrate with the first enzyme, thereby producing a first detectable signal associated with the first target nucleic acid molecule; contacting the sample with a second substrate for the second enzyme of the second set of label probes; reacting the second substrate with the second enzyme, thereby producing a second detectable signal associated with the second target nucleic acid molecule; and detecting the first detectable signal and the second detectable signal, thereby detecting the first and second target nucleic acids in the sample.

### BRIEF DESCRIPTION OF THE DRAWINGS

FIGS. 1A-1D show exemplary embodiments of the invention and basic principles of the invention. Abbreviations used: nucleic acid (NA); label probe (LP); enzyme (E); color (chromogenic) deposit (C). FIG. 1A shows direct binding of the label probe to the target nucleic acid. FIG. 1B shows the use of an intermediary complex. FIG. 1C shows reaction of a substrate with the first enzyme (E1) and generation of a first detectable signal associated with a first target nucleic acid (NA1). FIG. 1D shows the sequential reaction of a second substrate with the second enzyme (E2) and generation of a second detectable signal associated with a second target nucleic acid.

FIGS. 2A-2D show exemplary embodiments of the invention, in which sets of label probes are used, where multiple label probes within the set are capable of binding to different portions of the target nucleic acid and are labeled with the same enzyme. FIG. 2A shows direct binding of multiple copies of a set of label probes to different regions of the target nucleic acid. FIG. 2B shows binding of a set of label probes using an intermediary complex. FIG. 2C shows detection of two target nucleic acids with two sets of label probes, where the label probes bind directly to the target nucleic acid. FIG. 2D shows detection of two target nucleic acids, where the label probes are bound to the target nucleic acid using an intermediary complex.

FIGS. 3A and 3B show an exemplary embodiment of the invention, whereby a signal amplification system is used. FIG. 3A shows the use of amplifiers. FIG. 3B shows the use of amplifiers and preamplifiers.

FIG. 4 shows an exemplary embodiment of the invention, whereby a target probe (TP) is used to bind preamplifiers and amplifiers to the target nucleic acid.

FIG. 5 shows an exemplary embodiment of the invention using amplification with multiple target probes such as target probe pairs, whereby a pair of target probes are used to bind each preamplifier to the target nucleic acid. As shown, more than one preamplifier can be bound to the target nucleic acid by binding the preamplifiers to different regions of the target nucleic acid via the pair of target probes.

FIG. 6 shows detection of Apoe and Lgr5 mRNA in mouse brain tissue. Formalin-fixed paraffin-embedded mouse cerebellum tissue was analyzed for expression of Apoe and Lgr5 mRNA by simultaneous hybridization of label probes targeted to the respective nucleic acids. Chromogenic reactions were carried out sequentially for alkaline phosphatase using FastRed as substrate, followed by horseradish peroxidase. For horseradish peroxidase, the substrates used were DAB to produce a brown precipitate (FIG. 6A) or HRP-Green (42 Life Science, Bremerhaven Germany; P/N S-99054-1000) to produce a green precipitate. Leftward-pointing arrows indicate representative positions of red precipitate, and rightward-pointing arrows indicate representative cells containing brown precipitate (FIG. 6A) or green precipitate (FIG. 6B).

### DETAILED DESCRIPTION OF THE INVENTION

The present invention relates to a novel technique that allows simultaneous binding of two different enzyme-carrying reporter molecules to the corresponding targets, followed by sequential substrate reactions. The methods of the invention ensure that both reporter molecules bind to their respective targets with optimal efficiency. In addition, the assay protocol is simplified, and duration of the assay is shortened by eliminating a binding step and washing step.

Detecting multiple target nucleic acid molecules in the same sample in a single assay is important because it helps to reveal the biological relationship between different molecular biomarkers. As disclosed herein, an assay has been devised whereby at least two enzyme reporters are used to label at least two different nucleic acid targets, for example, RNA targets, in a single assay step. Thus, the methods of the invention allow multiplexed chromogenic detection of nucleic acids such as RNA or DNA in a simplified protocol.

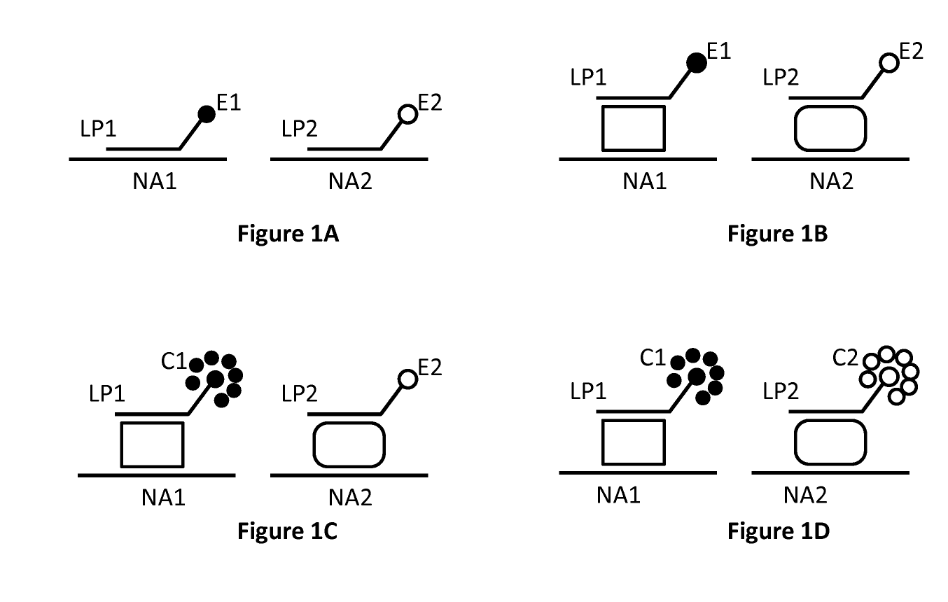

An overview of embodiments of the invention are described below. As illustrated in FIG. 1, a label probe (LP1) is conjugated to a chromogenic enzyme (E1). Another label probe (LP2) is conjugated to a different enzyme (E2). In the assay, at least one LP1 is specifically attached to target nucleic acid molecule (NA1) and at least one LP2 is specifically attached to NA2 in a single hybridization based assay step. The label probe can attach to the target directly by designing the sequence of the label probe to be complementary to a section of the target (FIG. 1A). Alternatively, the label probe can be attached to the target indirectly through an intermediary molecule or intermediary complex (FIG. 1B). Next, a substrate (S1) reacting with enzyme E1 is introduced, producing color deposit C1 at E1 site (FIG. 1C). This is followed by another assay step that introduces substrate S2 reacting with E2 to produce C2 (FIG. 1D). The same multiplexing scheme can be extended to more than two targets. For example, LP3, LP4, etc., that conjugate to E3, E4, etc., can be used to attach to NA3, NA4, etc., in a single assay step together with LP1 and LP2. Then S3, S4, etc., can react with E3, E4, etc., respectively, to produce C3, C4, etc., in sequential assay steps. Here the label probes can be made of oligos and the nucleic acid target can be DNA or RNA. The target nucleic acid molecules can reside inside cells and be detected in situ. The biological specimen where the cells reside can be formalin fixed and paraffin embedded (FFPE) tissue, fresh frozen tissue, freshly fixed tissue, cell lines, bacteria cells or white blood cells.

Although this method has been demonstrated for in situ detection, its applications can be extended to the detection of multiple target nucleic acid sequences that are outside cells but immobilized on a solid surface. Similarly, this method can also be used for fluorescent detection by using fluorescent substrates as S1, S2, and so forth.

In one embodiment of the invention, enzyme E1 and E2 are alkaline phosphatase (AP) and horseradish peroxidase (HRP), respectively. Correspondingly, S1 and S2 are Fast Red and 3,3-diaminobenzidine (DAB), respectively, which produce red and brown deposits to mark the presences of NA1 and NA2, respectively. S2 can alternatively be a green chromogen producing a green or black precipitate, thereby providing better contrast to the red precipitate produced by Fast Red.

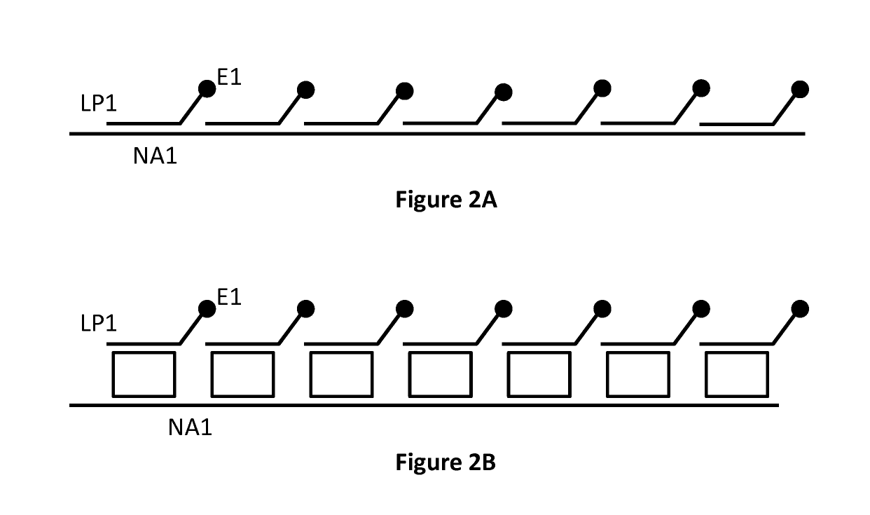

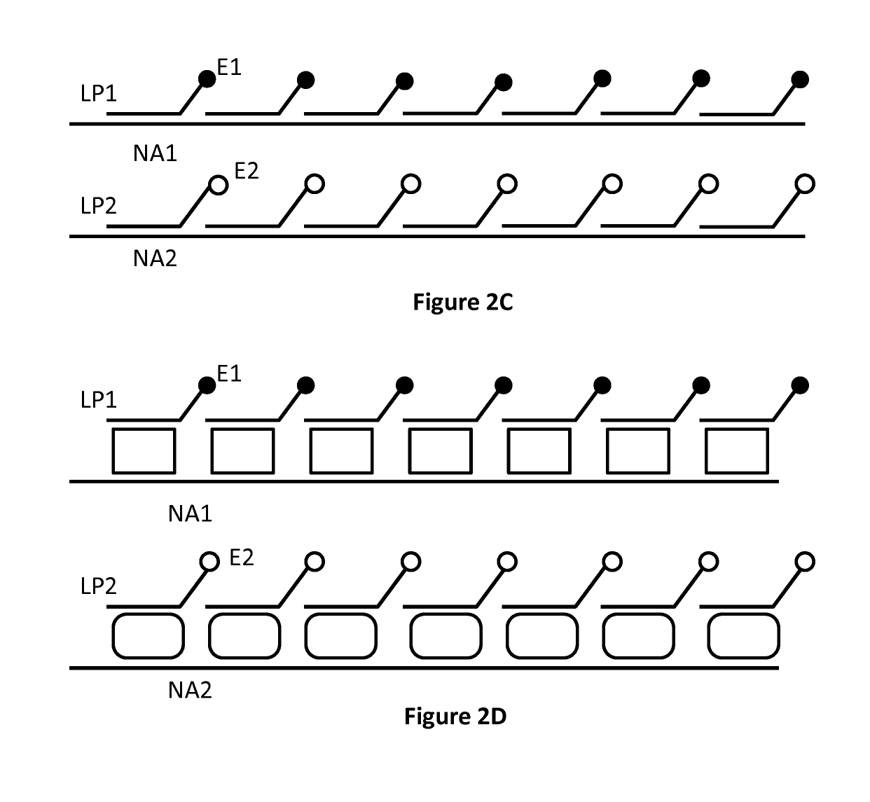

FIG. 2 shows another embodiment of the present invention. In this embodiment, LP comprises a set of short oligos that are hybridized directly to multiple sections along the target nucleic acid (FIG. 2A). In this case, the same enzyme E1 is attached to each of the multiple oligos hybrized along the target nucleic acid. Again, the LPs can also attach to the target indirectly through an intermediary molecule (FIG. 2B).

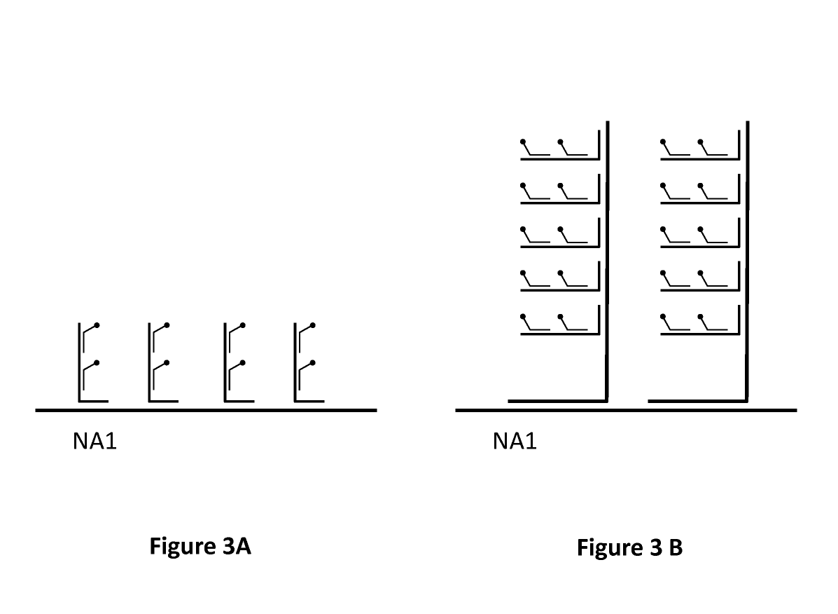

FIG. 3 illustrates a further embodiment of the scheme shown in FIG. 1B, where the intermediary molecule is an amplification system. Such an amplification system allows the binding of multiple copies of the LP to achieve signal amplification. Multiples of such amplifiers can be deployed along the target nucleic acid to further enhance the signal. Signal amplification can be particularly beneficial in this assay because it can be used to compensate for the reduced enzyme activity as a result of simultaneous reporter binding, as discussed herein.

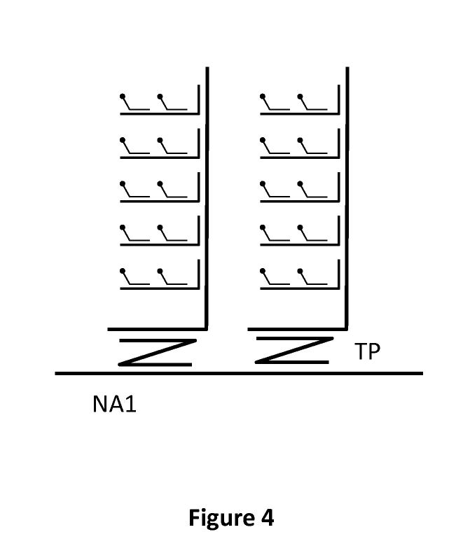

FIG. 4 shows a different embodiment compared to FIG. 3, where the amplification system is not directly bound to the target nucleic acid. Instead, it is attached to the target molecule through a target probe (TP), which has one section complimentary to a section of the target and another section complimentary to a section of the amplifier.

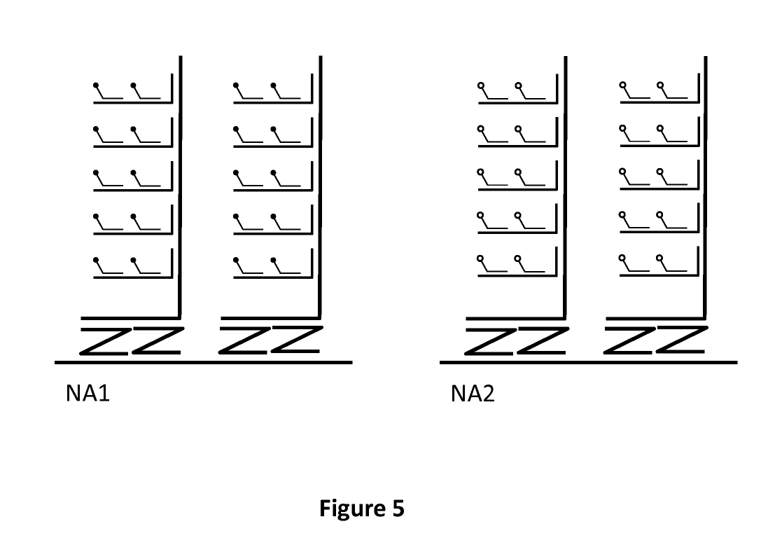

FIG. 5 shows a further implementation of the invention, where the target probe comprises a pair of short oligos that are designed to hybridize side by side between the target and the amplifier. When and only when both members of the pair are hybridized side by side to the target, the amplifier can be stably captured to the target to generate signal. Such a mechanism is referred to as collaborative hybridization, which serves to significantly enhance the specificity of the hybridization. This setup amplifies the true signal and suppresses the background at the same time, resulting in superior signal to noise ratio. FIG. 5 also shows the same approach applied to multiple nucleic acid targets in the assay.

In a further embodiment where the E2 is HRP and after E1 substrate has reacted and produced C1, tyramide conjugated with biotin is added to react with HRP, followed by addition of HRP-conjugated streptavidin, which binds to the biotin moiety of precipitated tyramide-biotin. This is followed by addition of HRP based chromogen substrate to produce color deposit. This tyramide based step provides additional amplification, which is more than sufficient to compensate for the loss of HRP activity during E1 substrate reaction.

As used herein, the term “nucleic acid,” or alternatively the term “polynucleotide,” refers to a polymer of nucleotide monomer units, as is well known to those skilled in the art. Exemplary nucleic acids include, for example, DNA and RNA, including mRNA, siRNA, hnRNA, genomic DNA, cDNA, or other well known forms of nucleic acids. The nucleic acid or polynucleotide can contain naturally occurring nucleotides, including well known naturally occurring nucleotide modifications, as well as non-naturally occurring nucleotides such as those made by chemical synthesis. Such modifications include, but are not limited to, peptide nucleic acids (PNAs), modified oligonucleotides, for example, oligonucleotides comprising nucleotides that are not typical to biological RNA or DNA, such as 2′-O-methylated oligonucleotides, and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like, as disclosed herein. The polynucleotide can be single-stranded or double-stranded.

As used herein, a “nucleic acid target” or “target nucleic acid” refers to a nucleic acid, or a region thereof, that is intended to be detected. One skilled in the art can readily determine target nucleic acids desired to be detected by methods of the invention.

As used herein, the term “label probe” refers to an entity that binds to a target molecule, directly or indirectly, and allows the target to be detected. A label probe (or “LP”) contains a nucleic acid binding portion that is typically a single-stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid, or it can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. Alternatively, the label probe can comprise at least one polynucleotide sequence that is complementary to a polynucleotide sequence in an amplifier, preamplifier, intermediary complex, or the like, as described herein. As used herein, a label probe comprising an enzyme label refers to a label probe comprising a nucleic acid binding portion such as an oligonucleotide and an enzyme that is coupled to the nucleic acid binding portion. As disclosed herein, the coupling of the enzyme to the nucleic acid binding portion can be covalent or through a high affinity binding interaction such as biotin/avidin or other similar high affinity binding molecules.

As used herein, a “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. In a particular embodiment of the invention, the label is an enzyme. Exemplary enzyme labels include, but are not limited to Horse Radish Peroxidase (HRP), Alkaline Phosphatase (AP), β-galactosidase, glucose oxidase, and the like, as well as various proteases. Other labels include, but are not limited to, fluorophores, Dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the invention, including detectable enzyme/substrate combinations (Pierce, Rockford Ill.; Santa Cruz Biotechnology, Dallas Tex.; Invitrogen, Carlsbad Calif.). In a particular embodiment of the invention, the enzyme can utilize a chromogenic or fluorogenic substrate to produce a detectable signal, as described herein.

As used herein, a “target probe” is a polynucleotide that is capable of hybridizing to a target nucleic acid and capturing or binding a label probe or intermediary complex molecule to that target nucleic acid. The target probe can hybridize directly to the label probe, or it can hybridize to one or more nucleic acids that in turn hybridize to the label probe; for example, the target probe can hybridize to an amplifier or a preamplifier in an intermediary complex. The target probe thus includes a first polynucleotide sequence that is complementary to a polynucleotide sequence of the target nucleic acid and a second polynucleotide sequence that is complementary to a polynucleotide sequence of the label probe, amplifier, preamplifier, or the like. The target probe is generally single-stranded so that the complementary sequence is available to hybridize with a corresponding target nucleic acid, label probe, amplifier or preamplifier.

As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, at least one target probe or nucleic acid bound to a target probe such as a preamplifier. For example, the amplifier can hybridize to at least one target probe and to a plurality of label probes, or to a preamplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688.

As used herein, a “preamplifier” is a molecule, typically a polynucleotide, that serves as an intermediate between one or more target probes and one or more amplifiers. Typically, the preamplifier hybridizes simultaneously to one or more target probes and to a plurality of amplifiers. Exemplary preamplifiers are described, for example, in U.S. Pat. Nos. 5,635,352, 5,681,697 and 7,709,198 and U.S. publications 2008/0038725 and 2009/0081688.

As used herein, an “intermediary complex” is a molecule, and can be a large, complex molecule, or an assembly of multiple molecules, that has one or more components, each containing a section capable of binding specifically to a section of the target nucleic acid and has another component or multiple components each containing one or multiple sections capable of binding to the label probe.

As used herein, “in situ hybridization” or “ISH” refers to a type of hybridization that uses a labeled complementary DNA or RNA strand, such as a probe, to bind to and localize a specific nucleic acid, such as DNA or RNA, in a sample, in particular a portion or section of tissue (in situ). The probe types can be double stranded DNA (dsDNA), single stranded DNA (ssDNA), single stranded complimentary RNA (sscRNA), messenger RNA (mRNA), micro RNA (miRNA), and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)).

The invention provides methods of detecting two or more target nucleic acids in a sample. In one embodiment, the method of the invention can include the steps of (a) contacting a sample with two or more label probes, wherein each label probe comprises a distinct enzyme label and targets a distinct nucleic acid target; (b) attaching or binding the two or more label probes to a first target nucleic acid and a second target nucleic acid in the sample by hybridization; (c) contacting the sample with a first substrate for the first enzyme of the first label probe; (d) reacting the first substrate with the first enzyme, thereby producing a first detectable signal associated with the first target nucleic acid molecule; (e) contacting the sample with a second substrate for the second enzyme of the second label probe; (f) reacting the second substrate with the second enzyme, thereby producing a second detectable signal associated with the second target nucleic acid molecule; and (g) detecting the first detectable signal and the second detectable signal, thereby detecting the first and second target nucleic acids in the sample.

An exemplary embodiment of the invention is shown in FIG. 1. In more detail, FIG. 1 shows an exemplary embodiment of the invention, where the detection of two nucleic acid targets (NA1 and NA2) is shown. It is understood by those skilled in the art that such nucleic acid targets are distinct from each other in that they are non-identical nucleic acid molecules having different nucleic acid sequences. Such distinct nucleic acid molecules can be distinguished from each other in methods of the invention by selection of appropriate regions of the nucleic acid target such that specific and distinguishable binding to the two nucleic acid targets can be achieved. Such a selection of appropriate regions and design of specific and selective reagents that bind to the target nucleic acids, in particular oligonucleotides or probes that specifically and selectively bind to a target nucleic acid, are well known to those skilled in the art (see Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999). A desired specificity can be achieved using appropriate selection of regions of a target nucleic acid as well as appropriate lengths of a binding agent such as an oligonucleotide or probe, and such selection methods are well known to those skilled in the art. Thus, one skilled in the art will readily understand and can readily determine appropriate reagents, such as oligonucleotides or probes, that can be used to target one particular target nucleic acid over another target nucleic acid.

In an embodiment of the invention as depicted in FIG. 1A, a sample containing the distinct target nucleic acids (NA1 and NA2) is contacted with two label probes. Each of the label probes is specific for a target nucleic acid. The specificity of a label probe for a target nucleic acid is achieved by (1) selection of a suitable nucleic acid target binding region that is an oligonucleotide complementary to the target nucleic acid, and (2) coupling the binding region comprising the oligonucleotide to a specific enzyme. The label probe thereby provides the association of a detectable agent, in this case an enzyme, with a particular nucleic acid target. As described in more detail herein, the enzymatic activity of the enzyme is utilized to generate a detectable signal. In the embodiment depicted in FIG. 1A, the label probe that targets a first nucleic acid target is distinct from a label probe that targets a second nucleic acid target. The label probe for the first nucleic acid target (LP1) comprises a binding region, that is, an oligonucleotide that is complementary to and can specifically and selectively hybridize with nucleic acid 1, and a first specific enzyme, whereas the second label probe (LP2) comprises a binding region, that is, an oligonucleotide that is complementary to and can specifically and selectively hybridize with nucleic acid 2 and a second specific enzyme, where the first and second specific enzymes are distinct from each other. For example, such specific and distinct enzymes can be selected from horseradish peroxidase, alkaline phosphatase, β-galactosidase and glucose oxidase, and the like, as disclosed herein. Thus, the label probe is designed such that it can specifically and selectively target a particular target nucleic acid and associate a distinguishable label to the target nucleic acid by utilizing distinct enzymes on label probes for different target nucleic acids.

As described herein, the methods of the invention utilize distinct enzymes that allow the association of a distinguishable detectable label with a target nucleic acid. Any of a number of enzymes can be utilized so long as the enzymatic activity can be detected. The enzyme thereby produces a detectable signal, which can be utilized to detect a target nucleic acid. Particularly useful detectable signals are chromogenic or fluorogenic signals. Accordingly, particularly useful enzymes for use in the invention include those for which a chromogenic or fluorogenic substrate is available. Such chromogenic or fluorgenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluoregenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present invention.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN) (4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis(2-amino-2-methyl-1,3-propanediol), 4-Methylumbelliferyl phosphate bis(cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford Ill.; Santa Cruz Biotechnology, Dallas Tex.; Invitrogen, Carlsbad Calif.; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord Calif.; biocare.net/products/detection/chromogens).

Biotin-avidin (or biotin-streptavidin) is a well known signal amplification system based on the facts that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized Horse Radish Peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, a lot of extra hapten molecules conjugated to tyramide can be introduced at the hybridization site in situ. Subsequently, the deposited tyramide-hapten molecules can be visualized directly or indirectly. Such a detection system is described in more detail in U.S. publication 2012/0100540.

In one embodiment of the invention, label probes are directly hybridized to the target nucleic acid molecules. This embodiment is exemplified in FIG. 1A. As discussed above, in such an embodiment the label probe itself comprises a nucleic acid target binding region, such as a complementary oligonucleotide, and binds directly to the target nucleic acid. Alternatively, the label probe can be bound to the target nucleic acid indirectly using an intermediary complex. Such an embodiment is depicted in FIG. 1B. In one such embodiment, the intermediary complex can be a single molecule such as a single nucleic acid molecule. For example, an intermediary complex that is a single molecule, that is, an intermediary molecule, can comprise a region that is capable of specifically and selectively attaching or binding to a target nucleic acid. This is similar to a label probe that is designed to attach or bind to a target nucleic acid, except that the binding to the nucleic acid target is mediated by the intermediary molecule. The intermediary molecule also can comprise a region that is capable of specifically and selectively binding to a label probe (see FIG. 1B). In such a case, the label probe and intermediary molecule are designed to have binding regions such as oligonucleotide regions that are complementary to each other. Such a configuration thereby associates the enzyme label to a target nucleic acid specifically and selectively through an intermediary molecule. As further depicted in FIG. 1A, a second target nucleic acid can be specifically and selectively associated with a distinct enzyme label through a second intermediary complex. The second intermediary complex comprises a first region that can specifically and selectively bind to the target nucleic acid and a second region that can specifically and selectively bind to the second label probe. Thus, the label probes can be bound to the target nucleic acid molecules by a first and second intermediary complex (see FIG. 1B). Further, the intermediary complex can comprise a molecule comprising a first region complementary to the target nucleic acid and the same or a different molecule comprising at least one second region complementary to the label probe. Further still, the intermediary complex can comprise an assembly of multiple molecules, of which one or more components each comprising one or more regions complementary to the target nucleic acid and one or more other components each comprising one or more regions complementary to the label probe. In this way, the intermediary complex can be an amplifying structure enabling multiple label probes to be attached to a region on target nucleic acid. In general, the intermediary complex comprises one or more nucleic acid molecules to take advantage of the specific and selective binding capabilities of nucleic acid interactions, as described herein.

As described herein, in a method of the invention a sample is contacted with two or more label probes, where each label probe comprises a distinct enzyme label and targets a distinct nucleic acid target. The label probes are bound to the target nucleic acid molecules by hybridization of complementary regions on the target nucleic acid and either the label probe or the intermediary complex, as discussed above. In a particular embodiment, the two or more label probes are contacted with the sample simultaneously so that the label probes are bound to the respective target nucleic acids in the same reaction. This is particularly useful in that association of label with each of the target nucleic acids can occur at the same time, thereby reducing assay time and reagent costs. This contrasts with previous methods, where hybridization reactions for each target nucleic acid are carried out sequentially.

As described herein, the label probe used in methods of the invention comprises a target nucleic acid binding region comprising an oligonucleotide, which is complementary to the target nucleic acid or complementary to a component of an intermediary complex that can bind to the target nucleic acid. In such a configuration of a label probe, the oligonucleotide portion is coupled to an enzyme. This differs from previous methods, where an enzyme is used to detect a nucleic acid target through an antibody, where the antibody is detected using well known immunodetection methods. The label probe configuration used in methods of the present invention are particularly useful because the enzyme label is coupled to an oligonucleotide that specifically and selectively binds to a target nucleic acid. The coupling of the enzyme to the oligonucleotide in the label probe can be covalent, using well known chemical coupling methods (see, for example, Hermanson, Bioconjugate Techniques, Academic Press, San Diego (1996)). Alternatively, a high affinity interaction such as biotin/avidin, where biotin or avidin is coupled to the oligonucleotide and enzyme in the label probe, can be used to couple the enzyme to the oligonucleotide in the label probe (see US publication 2012/0100540). It is understood that the coupling of the enzyme to the oligonucleotide the label probe is of sufficient affinity, such as through an interaction such as biotin/avidin, or is covalent such that the enzyme remains attached to the oligonucleotide throughout various reactions and assay conditions of the methods of the invention.

Once the label probes are bound to the target nucleic acids, either directly or via an intermediary complex, the sample is contacted with a first substrate for the first enzyme of the first label probe. Such a reaction is depicted in FIG. 1C. By using a first distinct enzyme and an appropriate substrate, a detectable signal can be generated that is associated with the target nucleic acid and is therefore distinct for the first target nucleic acid. Since the reaction conditions for a distinct enzyme are often different, and utilize a different substrate, the methods of the invention can be carried out such that a second substrate for the second distinct enzyme is added sequentially, after the reaction with the first enzyme has taken place. Optionally, the first enzyme can be inactivated prior to sequential addition of the second substrate for the second distinct enzyme. It is understood that, if the distinct enzymes are compatible, the first and second enzymatic reactions can be carried out concurrently. In a particular embodiment, the first enzymatic reaction is carried out and the detectable signal generated, followed sequentially by the second enzymatic reaction and production of the second detectable signal (see FIGS. 1C and 1D). The sequential steps can be carried out optionally with an intervening wash step, in particular if the reaction for the second enzyme is not compatible with reaction conditions for the first enzyme.

As described herein, distinct enzymes are associated with a target nucleic acid using label probes that target distinct nucleic acid targets. The association of a distinct enzyme with a target nucleic acid allows the use of the enzymatic activity of the enzyme to generate a detectable signal associated with a target nucleic acid. In particular, a chromogenic or fluorogenic substrate can be used to produce a detectable chromogenic or fluorescent signal associated with a target nucleic acid. Since distinct enzymes are associated with distinct target nucleic acids, the detectable signals are associated with the respective target nucleic acids, thereby allowing detection of the target nucleic acids. As described herein, well known methods such as microscopy or spectroscopy can be utilized to visualize chromogenic or fluorescent detectable signals associated with the respective target nucleic acids. In general, either chromogenic substrates or fluorogenic substrates will be utilized for a particular assay so that a single type of instrument can be used for detection of nucleic acid targets in the same sample.

In another embodiment, the invention provides a method of detecting two or more target nucleic acids. The method can comprise the step of (a) contacting a sample with two or more sets of label probes, wherein each set of label probes comprises a plurality of label probes, wherein the plurality of label probes within a set have the same enzyme label, and optionally wherein the plurality of label probes comprise label probes that bind to different regions of a target nucleic acid molecule; and wherein each set of label probes comprises a distinct enzyme label and targets a distinct nucleic acid target; (b) attaching or binding the two or more sets of label probes to a first target nucleic acid and a second target nucleic acid in the sample by hybridization; (c) contacting the sample with a first substrate for the first enzyme of the first set of label probes; (d) reacting the first substrate with the first enzyme, thereby producing a first detectable signal associated with the first target nucleic acid molecule; (e) contacting the sample with a second substrate for the second enzyme of the second set of label probes; (f) reacting the second substrate with the second enzyme, thereby producing a second detectable signal associated with the second target nucleic acid molecule; and (g) detecting the first detectable signal and the second detectable signal, thereby detecting the first and second target nucleic acids in the sample.

The methods can include the step of contacting a sample with two or more sets of label probes, wherein each set of label probes comprises a plurality of label probes, wherein the plurality of label probes within a set have the same enzyme label, and optionally wherein the plurality of label probes comprise label probes that attach or bind to different regions of a target nucleic acid molecule; and wherein each set of label probes comprises a distinct enzyme label and targets a distinct nucleic acid target; attaching or binding the two or more sets of label probes to two or more target nucleic acids, for example, at least a first target nucleic acid and a second target nucleic acid, or additional target nucleic acids, in the sample by hybridization; contacting the sample with a first substrate for the first enzyme of the first set of label probes; reacting the first substrate with the first enzyme, thereby producing a first detectable signal associated with the first target nucleic acid molecule; contacting the sample with a second substrate for the second enzyme of the second set of label probes; reacting the second substrate with the second enzyme, thereby producing a second detectable signal associated with the second target nucleic acid molecule; and detecting the first detectable signal and the second detectable signal, thereby detecting the first and second target nucleic acids in the sample.

An embodiment of the invention utilizing sets of probes is depicted in FIG. 2. As shown in FIG. 2A, a set of label probes is employed, where label probes within the set target different regions of the target nucleic acid. In general, a set of label probes for a target nucleic acid will hybridize to non-overlapping regions of the target nucleic acid. This allows each of the label probes within the set to bind independently of each other and non-competitively, thereby maximizing signal. Although the label probes within the set bind to different regions of the target nucleic acid, the same distinct enzyme is coupled to each of the label probes within the set. Thus, instead of having a single region of the target nucleic acid being bound by the label probe, as depicted in FIG. 1, multiple label probes are bound to the target nucleic acid. Since the same distinct enzyme is coupled to each of the label probes in the set, multiple copies of the enzyme are bound to the target nucleic acid. This configuration can therefore be used to substantially increase the detectable signal associated with the target nucleic acid.

As described above, the label probes in a set of label probes can be directly hybridized to the target nucleic acid molecules, as depicted in FIGS. 2A and 2C. Alternatively, the label probes can be bound to the target nucleic acid molecules by a first and second intermediary complex (see FIGS. 2B and 2D). As described herein, the intermediary complex can comprise a molecule comprising a first region complementary to the target nucleic acid and the same or another molecule comprising at least one second region complementary to the label probe (see FIGS. 2B and 2D). As depicted in FIGS. 2C and 2D, using sets of label probes provides for increased detectable signal to be associated with the respective target nucleic acids.

In still another embodiment, the methods of the invention can employ an intermediary complex, where the intermediary complex comprises multiple molecules rather than a single molecule. Such an intermediary complex is particularly useful for amplifying the detectable signal, providing higher sensitivity detection of target nucleic acids. Such methods for amplifying a signal are described, for example, in U.S. Pat. Nos. 5,635,352, 5,124,246, 5,710,264, 5,849,481, and 7,709,198, and U.S. publications 2008/0038725 and 2009/0081688, as well as WO 2007/001986 and WO 2012/054795.

In one embodiment utilizing an intermediary complex, the intermediary complex can comprise an amplifier or preamplifier. Such an embodiment is depicted in FIG. 3. In particular, FIG. 3A shows an intermediary complex, where an amplifier is used. With the use of an amplifier, which itself comprises a target nucleic acid binding region, multiple copies of the label probe can be attached to the target nucleic acid with a single amplifier. Although depicted in FIG. 3 as two label probes being bound to the amplifier, it is understood that this is merely exemplary and that multiple copies of the label probe can be bound to the amplifier, as desired. Thus, a plurality of label probes can be hybridized to one or more amplifiers. Similar to the embodiment shown in FIG. 2, multiple amplifiers can be used that bind to different regions of the target nucleic acid, thereby providing even greater signal amplification with each amplifier being bound to multiple copies of the label probe. As with other embodiments described herein, the label probes for targeting a particular nucleic acid target are coupled to the same enzyme.

In another embodiment depicted in FIG. 3B, the intermediary complex can comprise an amplifier and preamplifier. In this configuration, the preamplifier comprises a nucleic acid target binding region and multiple binding sites for amplifiers. Thus, a plurality of amplifiers can be hybridized to a preamplifier. Again the amplifiers can contain multiple binding sites for the label probe, and the label probes used for a particular nucleic acid target comprise the same enzyme so that the same distinct enzyme label is associated with the target nucleic acid. In a configuration using a preamplifier, the preamplifier is hybridized to the target nucleic acid.

In still another embodiment, the intermediary complex can comprise a target probe, amplifier or preamplifier. Such an embodiment is depicted in FIG. 4. Similar to the embodiment depicted in FIG. 3B, a preamplifier is used with multiple amplifiers bound, and multiple label probes are bound to the amplifiers. However, instead of the preamplifier binding directly to the nucleic acid target, a target probe is used. By using a target probe, the preamplifier is not required to have a nucleic acid target binding region, as depicted in FIG. 3B. Instead, the target probe is designed to comprise a binding region for the nucleic acid target. If more than one target probe is used, multiple target probes can be designed to bind to different regions of the target nucleic acid, as shown in FIG. 4. In such a configuration, the same preamplifier can be used for multiple binding events through the target probe instead of designing and generating different preamplifiers for targeting different regions of the target nucleic acid.

Thus, in a particular embodiment the intermediary complex can comprise a target probe, amplifier and preamplifier (see FIG. 4). In addition, a plurality of label probes can be hybridized to one or more amplifiers, a plurality of amplifiers can be hybridized to a preamplifier, and the preamplifier can be hybridized to a target probe.

In yet another embodiment, a configuration similar to that depicted in FIG. 4 can be used, where the intermediate complex comprises a target probe, preamplifier and amplifier. Rather than using a single target probe to bind the preamplifier to the target nucleic acid, two or more target probes are used to bind the preamplifier to the target nucleic acid (see FIG. 5). In general, the advantages of such a configuration can be realized using two target probes, that is, a pair of target probes to bind a preamplifier to the target nucleic acid. Such a configuration and its advantages for increasing sensitivity and decreasing background are described, for example, in U.S. Pat. No. 7,709,198, U.S. publications 2008/0038725 and 2009/0081688, and WO 2007/001986 and WO 2007/002006. U.S. Pat. No. 7,709,198 and U.S. publications 2008/0038725 and 2009/0081688 additionally describe details for selecting characteristics of the target probes, including target probe pairs, including length, orientation, hybridization conditions, and the like, as discussed below in more detail.

As disclosed herein, even though the figures generally depict detection of one or two target nucleic acids, it understood that the methods disclosed herein can be readily applied to one target nucleic acid or to multiplex detection of two or more nucleic acid targets, for example, 3, 4, 5, 6, 7, 8, 9, 10, or even larger numbers of distinct nucleic acid targets. All that is required is the availability of a suitable number of distinct enzymes for use as a distinct label for each of the target nucleic acids. Thus, the methods of the invention can be applied to detect multiple nucleic acid targets, in particular two or more nucleic acid targets, in a sample.

It is understood by those skilled in the art that any of a number of suitable samples can be used for detecting target nucleic acids using methods of the invention. The sample for use in methods of the invention will generally be a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy or blood sample. Such samples can be, but are not limited to, organs, tissues, tissue fractions and/or cells isolated from an organism such as a mammal. Exemplary biological samples include, but are not limited to, a cell lysate, a cell culture, including a primary cell culture, a cell line, a tissue, an organ, an organelle, a biological fluid, and the like. Additional biological samples include but are not limited to a skin sample, tissue biopsies, stool, bodily fluids, including blood and/or serum samples, saliva, semen, and the like.

The sample or biological specimen can be processed by any of a number of routine methods used for handling biological samples. For example, the cells can be used as isolated or processed, for example, as tissue slices or other tissue specimens, including fixing cells such as with formalin fixed and paraffin embedded (FFPE) tissue, fresh frozen tissue, cell lines, bacteria or yeast cells, white blood cells, and the like. Methods for processing tissue or cell samples for in situ hybridization are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)).

As disclosed herein, the methods of the invention provide for a convenient assay method to detect multiple target nucleic acids in the same sample. Methods have been previously described for detecting two DNA targets simultaneously, such as the two-color chromogenic ISH (CISH) assay from Ventana Medical System (Tucson Ariz.) (US2011/033176),), in which probes against two different genes are conjugated with digoxigenin (DIG) or dinitrophenyl (DNP), respectively. Then, anti-DIG and anti-DNP antibodies that are conjugated with different enzymes, such as horseradish peroxidase and alkaline phosphatase, are used to generate different color precipitates. While the assay allows visualization of the two targets, the visualization occurs using an antibody to direct the enzyme to the target nucleic acid.

A fundamental issue with the antibody based methods for detecting nucleic acid targets is that their specificity is limited by the specificity of antibody-epitope interaction as well as antibody-antibody cross-reactivity. This is particularly an issue for detecting short nucleic acid targets, such as RNA, because a high level of signal amplification is required, and any non-specific antibody binding and/or antibody cross-reactivity will have significant effects on the signal to background ratio. The present invention circumvents the limitations of such antibody-based methods because the label probe of the invention comprises a target nucleic acid binding region such as an oligonucleotide and a distinct enzyme that is coupled to the target nucleic acid binding region. As disclosed herein, by utilizing a label probe comprising nucleic acid to bind to the target nucleic acid, a high level of specificity and selectivity can be achieved by using appropriate in silico oligonucleotide design and by selecting and controlling hybridization conditions using well known methods. Such a system can be optimized for selectivity and specificity and minimized for “cross-reactivity” between nucleic acid targets. Furthermore, the methods of the invention provide a system whereby a distinct enzyme is targeted to a nucleic acid by way of nucleic acid hybridization, where the enzyme is directly coupled to a nucleic acid portion of the label probe. Therefore, the methods of the invention exhibit superior specificity over existing antibody-based simultaneous labeling systems for IHC or ISH. Although the methods of the invention can utilize an antibody as an intermediary to target an enzyme to a nucleic acid, in a particular embodiment of the invention, the methods of the invention use a label probe comprising a nucleic acid portion that binds to a complementary nucleic acid sequence and an enzyme, where direct coupling of the enzyme to the nucleic acid portion of the label probe is used. Such a method using a label probe comprising a nucleic acid portion and an enzyme specifically excludes the use of an antibody to target an enzyme to a nucleic acid. Thus, in an embodiment of the invention, the methods of detecting target nucleic acids are performed with the proviso that an antibody is not used to label a target nucleic acid with an enzyme.

The invention additionally provides kits comprising components for performing methods of the invention. Such kits can include, for example, label probes, amplifiers, preamplifiers, target probes, and the like. Such components can be designed as for any of the configurations disclosed herein. A kit of the invention can, for example, comprise label probes comprising distinct enzymes and suitable chromogenic or fluorogenic substrates for the respective enzymes for carrying out methods of the invention. The kits can contain non-target-specific components, which can be used for detection of any desired target nucleic acid, with a target specific binding component to be designed separately by the user of the kit. Alternatively, the kit can be designed to contain components that include binding agents for one or more particular target nucleic acids. The kit can additionally include instructions for using the components of the kit to carry out methods of the invention.

As described herein, embodiments of the invention can include the use of target probes. Such a configuration and its advantages for increasing sensitivity and decreasing background are described, for example, in U.S. Pat. No. 7,709,198, U.S. publications 2008/0038725 and 2009/0081688, and WO 2007/001986 and WO 2007/002006. U.S. Pat. No. 7,709,198 and U.S. publications 2008/0038725 and 2009/0081688 additionally describe details for selecting characteristics of the target probes, including target probe pairs, including length, orientation, hybridization conditions, and the like. One skilled in the art can readily identify suitable configurations based on the teachings U.S. Pat. No. 7,709,198, U.S. publications 2008/0038725 and 2009/0081688, and WO 2007/001986 and WO 2007/002006. In the description provided below, based on the disclosure herein and the teachings in U.S. Pat. No. 7,709,198, U.S. publications 2008/0038725 and 2009/0081688, and WO 2007/001986 and WO 2007/002006, one skilled in the art will understand that the term “label extender,” as used in these references and as discussed below, can be used interchangeably with the term “target probe,” as described herein and illustrated in the figures.

Briefly, a first general class of embodiments includes methods of detecting two or more nucleic acids of interest. In the methods, a sample comprising or suspected of comprising the nucleic acids of interest, two or more subsets of m label extenders, wherein m is at least two, and a label probe system are provided. Each subset of m label extenders is capable of hybridizing to one of the nucleic acids of interest. The label probe system comprises a label, and a component of the label probe system is capable of hybridizing simultaneously to at least two of the m label extenders in a subset.

Each nucleic acid of interest is hybridized to its corresponding subset of m label extenders, and the label probe system is hybridized to the m label extenders. The presence or absence of the label is then detected. Since the label is associated with the nucleic acid(s) of interest via hybridization of the label extenders and label probe system, the presence or absence of the label is correlated with the presence or absence of the nucleic acid(s) of interest and thus in the original sample.

The methods are useful for multiplex detection of nucleic acids, optionally highly multiplex detection. Thus, the two or more nucleic acids of interest (i.e., the nucleic acids to be detected) optionally comprise five or more, 10 or more, 20 or more, 30 or more, 40 or more, 50 or more, or even 100 or more nucleic acids of interest, while the two or more subsets of m label extenders comprise five or more, 10 or more, 20 or more, 30 or more, 40 or more, 50 or more, or even 100 or more subsets of m label extenders. In embodiments in which capture extenders, are used, a like number of subsets of capture extenders are provided.

The label probe system optionally includes a preamplifier, an amplification multimer, and a label probe, wherein the preamplifier is capable of hybridizing simultaneously to the at least two of the m label extenders and to a plurality of amplification multimers, and wherein the amplification multimer is capable of hybridizing simultaneously to the preamplifier and to a plurality of label probes.

As noted above, a component of the label probe system is capable of hybridizing simultaneously to at least two of the m label extenders in a subset. Typically, the component of the label probe system that hybridizes to the two or more label extenders is an amplification multimer or preamplifier. Thus, in one aspect, the label probe system comprises an amplification multimer or preamplifier, which amplification multimer or preamplifier is capable of hybridizing to the at least two label extenders, and the label probe system is hybridized to the m label extenders at a hybridization temperature, which hybridization temperature is greater than a melting temperature Tₘ of a complex between each individual label extender and the amplification multimer or preamplifier. The hybridization temperature is typically about 5° C. or more greater than the Tm, e.g., about 7° C. or more, about 10° C. or more, about 12° C. or more, about 15° C. or more, about 17° C. or more, or even about 20° C. or more greater than the Tm.

Each label extender typically comprises a polynucleotide sequence L-1 that is complementary to a polynucleotide sequence in the corresponding nucleic acid of interest and a polynucleotide sequence L-2 that is complementary to a polynucleotide sequence in the component of the label probe system. In one class of embodiments, the m label extenders in a subset each have L-1 5′ of L-2 or each have L-1 3′ of L-2. The length of L-2 can vary. For example, sequence L-2 can be 20 nucleotides or less in length, e.g., L-2 can be between 9 and 17 nucleotides in length or between 12 and 15 or between 13 and 15 nucleotides in length.

Another general class of embodiments provides methods of detecting one or more nucleic acids using a novel label extender configuration. In the methods, a sample comprising or suspected of comprising the nucleic acids of interest, one or more subsets of m label extenders, wherein m is at least two, and a label probe system are provided. Each subset of m label extenders is capable of hybridizing to one of the nucleic acids of interest. The label probe system comprises a label, and a component of the label probe system (e.g., a preamplifier or an amplification multimer) is capable of hybridizing simultaneously to at least two of the m label extenders in a subset. Each label extender comprises a polynucleotide sequence L-1 that is complementary to a polynucleotide sequence in the corresponding nucleic acid of interest and a polynucleotide sequence L-2 that is complementary to a polynucleotide sequence in the component of the label probe system, and the at least two label extenders (e.g., the m label extenders in a subset) each have L-1 5′ of L-2 or each have L-1 3′ of L-2.

Each nucleic acid of interest is hybridized to its corresponding subset of m label extenders, and the label probe system is hybridized to the m label extenders at a hybridization temperature. The hybridization temperature is greater than a melting temperature Tm of a complex between each individual label extender and the component of the label probe system. Since the label is associated with the nucleic acid(s) of interest via hybridization of the label extenders and label probe system and the presence or absence of the nucleic acid(s) of interest can be determined.

Yet another general class of embodiments provides methods of capturing a label to a first nucleic acid of interest in a multiplex assay in which two or more nucleic acids of interest are to be detected. In the methods, a sample comprising the first nucleic acid of interest and also comprising or suspected of comprising one or more other nucleic acids of interest is provided. A first subset of m label extenders, wherein m is at least two, and a label probe system comprising the label are also provided. The first subset of m label extenders is capable of hybridizing to the first nucleic acid of interest, and a component of the label probe system is capable of hybridizing simultaneously to at least two of the m label extenders in the first subset. The first nucleic acid of interest is hybridized to the first subset of m label extenders, and the label probe system is hybridized to the m label extenders, thereby capturing the label to the first nucleic acid of interest.

Essentially all of the features noted tier the embodiments above apply to these methods as well, as relevant; for example, with respect to configuration of the label extenders, number of label extenders per subset, composition of the label probe system, type of label, number of nucleic acids of interest, source of the sample and/or nucleic acids, and/or the like.

Yet another general class of embodiments provides methods of capturing a label to a nucleic acid of interest. In the methods, m label extenders, wherein m is at least two, are provided. The m label extenders are capable of hybridizing to the nucleic acid of interest. A label probe system comprising the label is also provided. A component of the label probe system is capable of hybridizing simultaneously to at least two of the m label extenders. Each label extender comprises a polynucleotide sequence L-1 that is complementary to a polynucleotide sequence in the nucleic acid of interest and a polynucleotide sequence L-2 that is complementary to a polynucleotide sequence in the component of the label probe system, and the m label extenders each have L-1 5′ of L-2 or wherein the m label extenders each have L-1 3′ of L-2. The nucleic acid of interest is hybridized to the m label extenders, and the label probe system is hybridized to the m label extenders at a hybridization temperature, thereby capturing the label to the nucleic acid of interest. Preferably, the hybridization temperature is greater than a melting temperature Tm of a complex between each individual label extender and the component of the label probe system.

A “label extender” or “LE” is a polynucleotide that is capable of hybridizing to a nucleic acid of interest and to a label probe system. The label extender typically has a first polynucleotide sequence L-1, which is complementary to a polynucleotide sequence of the nucleic acid of interest, and a second polynucleotide sequence L-2, which is complementary to a polynucleotide sequence of the label probe system (e.g., L-2 can be complementary to a polynucleotide sequence of an amplification multimer, a preamplifier, a label probe, or the like). The label extender is preferably single-stranded.

A “label probe system” comprises one or more polynucleotides that collectively comprise a label and at least two polynucleotide sequences M-1, each of which is capable of hybridizing to a label extender. The label provides a signal, directly or indirectly. Polynucleotide sequence M-1 is typically complementary to sequence L-2 in the label extenders. The at least two polynucleotide sequences M-1 are optionally identical sequences or different sequences. The label probe system can include a plurality of label probes (e.g., a plurality of identical label probes) and an amplification multimer; it optionally also includes a preamplifier or the like, or optionally includes only label probes, for example.

An “amplification multimer” is a polynucleotide comprising a plurality of polynucleotide sequences M-2, typically (but not necessarily) identical polynucleotide sequences M-2. Polynucleotide sequence M-2 is complementary to a polynucleotide sequence in the label probe. The amplification multimer also includes at least one polynucleotide sequence that is capable of hybridizing to a label extender or to a nucleic acid that hybridizes to the label extender, e.g., a preamplifier. For example, the amplification manner optionally includes at least one (and preferably at least two) polynucleotide sequence(s) M-1, optionally identical sequences M-1; polynucleotide sequence M-1 is typically complementary to polynucleotide sequence L-2 of the label extenders. Similarly, the amplification multimer optionally includes at least one polynucleotide sequence that is complementary to a polynucleotide sequence in a preamplifier. The amplification multimer can be, e.g., a linear or a branched nucleic acid. As noted for all polynucleotides, the amplification multimer can include modified nucleotides and/or nonstandard internucleotide linkages as well as standard deoxyribonucleotides, ribonucleotides, and/or phosphodiester bonds. Suitable amplification multimers are described, for example, in U.S. Pat. No. 5,635,352, U.S. Pat. No. 5,124,246, U.S. Pat. No. 5,710,264, and U.S. Pat. No. 5,849,481.

Signal amplification begins with the binding of the LEs to the target mRNA. An amplification multimer is then typically hybridized to the LEs. The amplification multimer has multiple copies of a sequence that is complementary to a label probe (it is worth noting that the amplification multimer is typically, but not necessarily, a branched-chain nucleic acid; for example, the amplification multimer can be a branched, forked, or comb-like nucleic acid or a linear nucleic acid). A label, for example, alkaline phosphatase, is covalently attached to each label probe.

In the preceding example, the amplification multimer and the label probes comprise a label probe system. In another example, the label probe system also comprises a preamplifier, e.g., as described in U.S. Pat. No. 5,635,352 and U.S. Pat. No. 5,681,697, which further amplifies the signal from a single target mRNA. In yet another example, the label extenders hybridize directly to the label probes and no amplification multimer or preamplifier is used, so the signal from a single target mRNA molecule is only amplified by the number of distinct label extenders that hybridize to that mRNA.

Among other aspects, the present invention provides multiplex bDNA assays that can be used for simultaneous detection of two or more target nucleic acids. Similarly, one aspect of the present invention provides bDNA assays, singleplex or multiplex, that have reduced background from nonspecific hybridization events.

In general, in the assays of the invention, two or more label extenders are used to capture a single component of the label probe system (e.g., a preamplifier or amplification multimer). The assay temperature and the stability of the complex between a single LE and the component of the label probe system (e.g., the preamplifier or amplification multimer) can be controlled such that binding of a single LE to the component is not sufficient to stably associate the component with a nucleic acid to which the LE is bound, whereas simultaneous binding of two or more LEs to the component can capture it to the nucleic acid. Requiring such cooperative hybridization of multiple LEs for association of the label probe system with the nucleic acid(s) of interest results in high specificity and low background from cross-hybridization of the LEs with other, non-target nucleic acids.

For an assay to achieve high specificity and sensitivity, it preferably has a low background, resulting, e.g., from minimal cross-hybridization. Such low background and minimal cross-hybridization are typically substantially more difficult to achieve in a multiplex assay than a single-plex assay, because the number of potential nonspecific interactions are greatly increased in a multiplex assay due to the increased number of probes used in the assay (e.g., the greater number of CEs and LEs). Requiring multiple simultaneous LE-label probe system component interactions for the capture of the label probe system to a target nucleic acid minimizes the chance that nonspecific capture will occur, even when some nonspecific CE-LE or LE-CP interactions, for example, do occur. This reduction in background through minimization of undesirable cross-hybridization events thus facilitates multiplex detection of the nucleic acids of interest.

The label probe system optionally includes an amplification multimer and a plurality of label probes, wherein the amplification multimer is capable of hybridizing to the label extenders and to a plurality of label probes. In another aspect, the label probe system includes a preamplifier, a plurality of amplification multimers, and a plurality of label probes, wherein the preamplifier hybridizes to the label extenders, and the amplification multimers hybridize to the preamplifier and to the plurality of label probes. As another example, the label probe system can include only label probes, which hybridize directly to the label extenders. In one class of embodiments, the label probe comprises the label, e.g., a covalently attached label. In other embodiments, the label probe is configured to bind a label; for example, a biotinylated label probe can bind to a streptavidin-associated label.

Each label extender typically includes a polynucleotide sequence L-1 that is complementary to a polynucleotide sequence in the corresponding nucleic acid of interest and a polynucleotide sequence L-2 that is complementary to a polynucleotide sequence in the component of the label probe system (e.g., the preamplifier or amplification multimer). It will be evident that the amount of overlap between each individual label extender and the component of the label probe system (i.e., the length of L-2 and M-1) affects the Tm of the complex between the label extender and the component, as does, e.g., the GC base content of sequences L-2 and M-1. Optionally, all the label extenders have the same length sequence L-2 and/or identical polynucleotide sequences L-2. Alternatively, different label extenders can have different length and/or sequence polynucleotide sequences L-2. It will also be evident that the number of label extenders required for stable capture of the component to the nucleic acid of interest depends, in part, on the amount of overlap between the label extenders and the component (i.e., the length of L-2 and M-1).

Stable capture of the component of the label probe system by the at least two label extenders, e.g., while minimizing capture of extraneous nucleic acids, can be achieved, for example, by balancing the number of label extenders that bind to the component, the amount of overlap between the label extenders and the component (the length of L-2 and M-1), and/or the stringency of the conditions under which the label extenders and the component are hybridized.

Appropriate combinations of the amount of complementarity between the label extenders and the component of the label probe system, number of label extenders binding to the component, and stringency of hybridization can, for example, be determined experimentally by one of skill in the art. For example, a particular number of label extenders and a particular set of hybridization conditions can be selected, while the number of nucleotides of complementarity between the label extenders and the component is varied until hybridization of the label extenders to a nucleic acid captures the component to the nucleic acid while hybridization of a single label extender does not efficiently capture the component. Stringency can be controlled, for example, by controlling the formamide concentration, chaotropic salt concentration, salt concentration, pH, organic solvent content, and/or hybridization temperature.

As noted, the Tm of any nucleic acid duplex can be directly measured, using techniques well known in the art. For example, a thermal denaturation curve can be obtained for the duplex, the midpoint of which corresponds to the Tm. It will be evident that such denaturation curves can be obtained under conditions having essentially any relevant pH, salt concentration, solvent content, and/or the like.

Typically, the component of the label probe system (e.g., the amplification multimer or preamplifier) is capable of hybridizing simultaneously to two of the m label extenders in a subset, although it optionally hybridizes to three, four, or more of the label extenders. In one class of embodiments, e.g., embodiments in which two (or more) label extenders bind to the component of the label probe system, sequence L-2 is 20 nucleotides or less in length. For example, L-2 can be between 9 and 17 nucleotides in length, e.g., between 12 and 15 nucleotides in length, between 13 and 15 nucleotides in length, or between 13 and 14 nucleotides in length. As noted, m is at least two, and can be at least three, at least five, at least 10, or more. m can be the same or different from subset to subset of label extenders.

The label extenders can be configured in any of a variety ways. For example, the two label extenders that hybridize to the component of the label probe system can assume a cruciform arrangement, with one label extender having L-1 5′ of L-2 and the other label extender having L-1 3′ of L-2. Unexpectedly, however, a configuration in which either the 5′ or the 3′ end of both label extenders hybridizes to the nucleic acid while the other end binds to the component yields stronger binding of the component to the nucleic acid than does a cruciform arrangement of the label extenders. Thus, in one class of embodiments, the at least two label extenders (e.g., the m label extenders in a subset) each have L-1 5′ of L-2 or each have L-1 3′ of L-2. For example, L-1, which hybridizes to the nucleic acid of interest, can be at the 5′ end of each label extender, while L-2, which hybridizes to the component of the label probe system, is at the 3′ end of each label extender (or vice versa). L-1 and L-2 are optionally separated by additional sequence. In one exemplary embodiment, L-1 is located at the 5′ end of the label extender and is about 20-30 nucleotides in length, L-2 is located at the 3′ end of the label extender and is about 13-14 nucleotides in length, and L-1 and L-2 are separated by a spacer (e.g., 5 Ts).

The invention also provides methods of the invention as disclosed herein, with the additional embodiments described below. The steps of such a method can include, for example, providing a sample comprising the cell, which cell comprises or is suspected of comprising one or more nucleic acid targets; providing for each nucleic acid target a label probe system comprising label; providing for each nucleic acid target a label extender system comprising two or more different label extenders, wherein each of the two or more label extenders comprises a section T complementary to a section on the nucleic acid target and a section L complementary to a section on a component of the label probe system, and wherein the T sections are complementary to non-overlapping regions of the nucleic acid target, and the L sections are complementary to non-overlapping regions of the component of the label probe system; hybridizing, in the cell, the label extenders system to the nucleic acid target, when present in the cell, thereby providing hybridization of two or more different label extenders to a single copy of the nucleic acid target molecule; capturing, in the cell and in the presence of cellular non-target nucleic acids, the label probe system to the label extender system hybridized to the nucleic acid target molecule, thereby capturing the label to the nucleic acid target, wherein the capturing occurs by simultaneously hybridizing the at least two different label extenders to a single molecule of the component of the label probe system that is complementary to the label extenders; and detecting a signal from the label.

An embodiment can include hybridizing the at least two different label extenders to the single molecule of the component of the label probe system that is complementary to the label extenders is performed at a hybridization temperature that is greater than a melting temperature Tm of a complex between each individual label extender and the component of the label probe system. In another embodiment, the at least two different label extenders hybridize to adjacent sections on the nucleic acid target.

In still another embodiment, the methods of the invention disclosed herein can include additionally a method of detecting an individual cell of a specified type by detecting one or more nucleic acid targets within the individual cell. The steps of such a method can include, for example, providing a sample comprising a mixture of cell types, which mixture comprises or is suspected of comprising at least one cell of the specified type which comprises one or more nucleic acid targets; providing for each nucleic acid target a label probe system comprising label; providing for each nucleic acid target a label extender system comprising two or more different label extenders, wherein each of the two or more label extenders comprises a section T complementary to a section on the nucleic acid target and a section L complementary to a section on a component of the label probe system, and wherein the T sections are complementary to non-overlapping regions of the nucleic acid target, and the L sections are complementary to non-overlapping regions of the component of the label probe system; hybridizing, in the cell, the label extender system to the nucleic acid target, when present in the cell, thereby providing hybridization of two or more different label extenders to a single copy of the nucleic acid target molecule; capturing, in the cell and in the presence of cellular non-target nucleic acids, the label probe system to the label extender system hybridized to the nucleic acid target molecule, thereby capturing the label to the nucleic acid target, wherein the capturing occurs by simultaneously hybridizing the at least two different label extenders to a single molecule of the component of the label probe system that is complementary to the label extenders; detecting a signal from the label; correlating the signal detected from the cell with the presence, absence, or amount of the one or more nucleic acid targets in the cell; and identifying the cell as being of the specified type based on detection of the presence, absence, or amount of the one or more nucleic acid targets within the cell.

An embodiment can include hybridizing the at least two different label extenders to the single molecule of the component of the label probe system is performed at a hybridization temperature that is greater than a melting temperature Tm of a complex between each individual label extender and the label probe, amplifier, or preamplifier. Additionally, the at least two different label extenders hybridize to adjacent sections on the nucleic acid target. Also, hybridizing the two or more label extenders to the single molecule of the nucleic acid target can be performed at a hybridization temperature that is greater than a melting temperature Tm of a complex between each individual label extender and the nucleic acid target. Another embodiment can further comprise providing one or more blocking probes capable of hybridizing to regions of the nucleic acid target not occupied by the label extenders. In still another embodiment, each hybridizing or capturing step is accomplished for all the nucleic acid targets at the same time. In yet a further embodiment, the one or more nucleic acid targets are independently selected from the group consisting of a DNA, a chromosomal DNA, an RNA, an mRNA, a microRNA, a ribosomal RNA, a nucleic acid endogenous to the cell, and a nucleic acid introduced to or expressed in the cell by infection of the cell with a pathogen. In still another embodiment, the one or more nucleic acid targets comprise a first nucleic acid target comprising a first region of an mRNA and a second nucleic acid target comprising a second region of the same mRNA. In a further embodiment, the one or more nucleic acid targets can comprise a reference nucleic acid, and wherein the method comprises normalizing the signal of the one or more nucleic acid targets to the signal of the reference nucleic acid. Still a further embodiment can comprise the step of correlating the intensity of the signal of each nucleic acid target with a quantity of the corresponding nucleic acid target present in the cell. The sample comprising the cell can be derived from a bodily fluid, from blood or a tissue section, as disclosed herein. The cell can be in suspension during the hybridizing, capturing, and/or detecting steps. The cell can be a circulating tumor cell.

In a particular embodiment, each label extender is in a “Z” configuration, wherein the Z configuration comprises the T section of the label extender at the 5′ end and the L section of the label extender at the 3′ end, or the T section of the label extender at the 3′ end and the L section of the label extender at the 5′ end. In another embodiment, the label probe system comprises (A) (i) a label probe comprising one or more labels, or (B) (i) one or more label probes and (ii) an amplifier capable of hybridizing to one or more of the label probes, or (C) (i) one or more label probes, (ii) one or more amplifiers, and (iii) a preamplifier capable of hybridizing to one or more of the amplifiers. In a further embodiment, the label is captured to the nucleic acid target molecule by hybridization of the label probe to the at least two different label extenders hybridized to the nucleic acid target molecule. In still a further embodiment, the label is captured to the nucleic acid target molecule by hybridization of the one or more label probes to the amplifier molecule hybridized to the at least two different label extenders hybridized to the nucleic acid target molecule. In yet another embodiment, the label is captured to the nucleic acid target molecule by hybridization of the one or more label probes to the one or more amplifiers hybridized to the preamplifier molecule hybridized to the at least two different label extenders hybridized to the nucleic acid target molecule. In a still further embodiment, a plurality of label probe systems are provided for each target nucleic acid and wherein two or more label probe systems are captured to the target nucleic acid by using the label extender system comprising two or more different label extenders and wherein each label extender in the label extender system comprises a T section that is complementary to non-overlapping regions of the nucleic acid target. In yet another embodiment, three or more label probe systems are captured to the target nucleic acid by the label extender system.

The methods of the invention can additionally include further aspects as described below. For example, the methods can further include a method of detecting one or more nucleic acids of interest, the method comprising: a) providing a sample comprising or suspected of comprising the one or more nucleic acids of interest; b) labeling those nucleic acids of interest present in the sample using the methods disclosed herein; c) providing one or more subsets of m label extenders, wherein m is at least two, wherein each subset of m label extenders is capable of hybridizing to one of the nucleic acids of interest; d) providing a label probe system comprising a label, wherein a component of the label probe system is capable of hybridizing simultaneously to at least two of the m label extenders in a subset, wherein each of said at least two label extenders comprises a polynucleotide sequence L-1 that is complementary to a polynucleotide sequence in the corresponding nucleic acid of interest and comprises a polynucleotide sequence L-2 that is complementary to a polynucleotide sequence in the component of the label probe system, wherein said at least two label extenders all have L-1 5′ of L-2 or all have L-1 3′ of L-2, and wherein the m label extenders in the subset are complementary to nonoverlapping sequences in the corresponding nucleic acid of interest; e) hybridizing any nucleic acid of interest captured to its corresponding subset of m label extenders; f) hybridizing the label probe system to the m label extenders at a hybridization temperature, which hybridization temperature is greater than a melting temperature Tm of a complex between each individual label extender and the component of the label probe system; and g) detecting the presence or absence of the label on the target nucleic acid.

In a particular embodiment, the one or more nucleic acids of interest comprise five or more, 10 or more, 20 or more, 30 or more, 40 or more, or 50 or more different nucleic acids of interest, which different nucleic acids of interest are different nucleic acid molecules; and wherein the one or more subsets of m label extenders comprise five, or more, 10 or more, 20 or more, 30 or more, 40 or more, or 50 or more different subsets of m label extenders. In another embodiment, the label probe system comprises a preamplifier, an amplification multimer and a label probe; wherein the preamplifier is capable of hybridizing simultaneously to the at least two of the m label extenders and to a plurality of amplification multimers: wherein the amplification multimer is capable of hybridizing simultaneously to the preamplifier and to a plurality of label probes; and wherein the label probe comprises the label. In another embodiment, one or more nucleic acids of interest comprise two or more different nucleic acids of interest, which different nucleic acids of interest are different nucleic acid molecules, and wherein the one or more subsets of ill label extenders comprise two or more different subsets of m label extenders. In a particular embodiment, the label probe system comprises an amplification multimer or preamplifier, which amplification multimer or preamplifier is capable of hybridizing to the at least two label extenders.

In an embodiment, the hybridization temperature is about 5° C. or more greater than the Tm of a complex between each individual label extender and the component of the label probe system. Alternatively, the hybridization temperature is about 7° C. or more, about 10° C. or more, about 12° C. or more, about 15° C. or more, about 17° C. or more, or about 20° C. or more greater than the Tm of a complex between each individual label extender and the component of the label probe system. In another embodiment, the polynucleotide sequence L-2 is 20 nucleotides or less in length. Alternatively, L-2 is between 9 and 17 nucleotides in length. Alternatively, L-2 is between 13 and 15 nucleotides in length.

In another embodiment, each of said at least two label extenders has L-1 at its 5′ end and L-2 at its 3′ end. In still another embodiment, each of the at least two label extenders has L-1 at its 3′ end and L-2 at its 5′ end. In still another embodiment, the m label extenders in a subset all have L-1 5′ of L-2 or all have L-1 3′ of L-2. In another embodiment, the label probe system comprises an amplification multimer and a label probe; wherein the amplification multimer is capable of hybridizing simultaneously to the at least two of the m label extenders and to a plurality of label probes; and wherein the label probe comprises the label. In another embodiment, the label probe system comprises a label probe; wherein the label probe is capable of hybridizing simultaneously to the at least two of the m label extenders; and wherein the label probe comprises the label. In another embodiment, the component of the label probe system is capable of hybridizing simultaneously to two of the m label extenders in a subset.

A method of the invention can further involve capturing a label to a nucleic acid of interest, the method comprising: a) providing m label extenders, wherein in is at least two, wherein the m label extenders are capable of hybridizing to nonoverlapping sequences in the nucleic acid of interest; b) providing a label probe system comprising the label, wherein a component of the label probe system is capable of hybridizing simultaneously to at least two of the m label extenders, wherein each of said at least two label extenders comprises a polynucleotide sequence L-1 that is complementary to a polynucleotide sequence in the nucleic acid of interest and comprises a polynucleotide sequence L-2 that is complementary to a polynucleotide sequence in the component of the label probe system, and wherein said at least two label extenders all have L-1 5′ of L-2 or all have L-1 3′ of L-2; c) hybridizing the nucleic acid of interest to the m label extenders; and d) hybridizing the label probe system to the in label extenders at a hybridization temperature, which hybridization temperature is greater than a melting temperature Tm of a complex between each individual label extender and the component of the label probe system, thereby capturing the label to the nucleic acid of interest.

In an embodiment, the in label extenders all have L-1 5′ of L-2 or all have L-1 3′ of L-2. In an embodiment, the label probe system comprises an amplification multimer or preamplifier, which amplification multimer or preamplifier is capable of hybridizing to the at least two of the m label extenders. In an embodiment, the label probe system comprises a preamplifier, an amplification multimer and a label probe; wherein the preamplifier is capable of hybridizing simultaneously to the at least two of the m label extenders and to a plurality of amplification multimers; wherein the amplification multimer is capable of hybridizing simultaneously to the preamplifier and to a plurality of label probes; and wherein the label probe comprises the label. In an embodiment, the label probe system comprises an amplification multimer and a label probe; wherein the amplification multimer is capable of hybridizing simultaneously to the at least two of the m label extenders and to a plurality of label probes; and wherein the label probe comprises the label. In an embodiment, the label probe system comprises a label probe; wherein the label probe is capable of hybridizing simultaneously to the at least two of the m label extenders; and wherein the label probe comprises the label. In an embodiment, the component of the label probe system is capable of hybridizing simultaneously to two of the m label extenders. In an embodiment, the hybridization temperature is about 5° C. or more greater than the Tm of a complex between each individual label extender and the component of the label probe system. In an embodiment, the hybridization temperature is about 7° C. or more, about 10° C. or more, about 12° C. or more, about 15° C. or more, about 17° C. or more, or about 20° C. or more greater than the Tm of a complex between each individual label extender and the component of the label probe system. In an embodiment, the polynucleotide sequence L-2 is 20 nucleotides or less in length. In an embodiment, L-2 is between 9 and 17 nucleotides in length. In an embodiment, L-2 is between 13 and 15 nucleotides in length. In an embodiment, each of the at least two label extenders has L-1 at its 5′ end and L-2 at its 3′ end. In an embodiment, each of the at least two label extenders has L-1 at its 3′ end and L-2 at its 5′ end.

A further embodiment can include a method of detecting one or more nucleic acids of interest, the method comprising: a) providing a sample comprising the one or more nucleic acids of interest; b) labeling the nucleic acids of interest using the methods disclosed herein; c) providing a label probe system comprising a label; d) for each of the nucleic acids of interest, providing a subset of m label extenders, wherein m is at least two, wherein the subset of m label extenders is capable of hybridizing to that nucleic acid of interest, wherein a component of the label probe system is capable of hybridizing simultaneously to at least two of the m label extenders in the subset, wherein each of said at least two label extenders comprises a polynucleotide sequence L-1 that is complementary to a polynucleotide sequence in the nucleic acid of interest and comprises a polynucleotide sequence L-2 that is complementary to a polynucleotide sequence in the component of the label probe system, and wherein said at least two label extenders all have L-1 5′ of L-2 or all have L-1 3′ of L-2; e) hybridizing each nucleic acid of interest to its corresponding subset of m label extenders; f) hybridizing the label probe system to the label extenders at a hybridization temperature, which hybridization temperature is greater than a melting temperature Tm of a complex between each individual label extender and the component of the label probe system, whereby, for each of the nucleic acids of interest, a single copy of each of said at least two label extenders hybridizes simultaneously to a single copy of the nucleic acid of interest and to a single copy of the component of the label probe system; and g) detecting the presence or absence of the label on the target nucleic acid.

In yet another embodiment, a method can include capturing a label to a nucleic acid of interest, the method comprising: a) providing m label extenders, wherein m is at least two, wherein the m label extenders are capable of hybridizing to the nucleic acid of interest; b) providing a label probe system comprising the label, wherein a component of the label probe system is capable of hybridizing simultaneously to at least two of the m label extenders, wherein each of said at least two label extenders comprises a polynucleotide sequence L-1 that is complementary to a polynucleotide sequence in the nucleic acid of interest and comprises a polynucleotide sequence L-2 that is complementary to a polynucleotide sequence in the component of the label probe system, and wherein said at least two label extenders all have L-1 5′ of L-2 or all have L-1 3′ of L-2; c) hybridizing the nucleic acid of interest to the m label extenders; and d) hybridizing the label probe system to the m label extenders at a hybridization temperature, which hybridization temperature is greater than a melting temperature Tm of a complex between each individual label extender and the component of the label probe system, whereby a single copy of each of said at least two label extenders hybridizes simultaneously to a single copy of the nucleic acid of interest and to a single copy of the component of the label probe system, thereby capturing the label to the nucleic acid of interest.

It is understood that modifications which do not substantially affect the activity of the various embodiments of this invention are also provided within the definition of the invention provided herein. Accordingly, the following examples are intended to illustrate but not limit the present invention.

### EXAMPLE I

### In Situ Detection of Nucleic Acid Targets in Mouse Brain Tissue

This example describes the in situ detection of nucleic acid targets in mouse brain tissue.

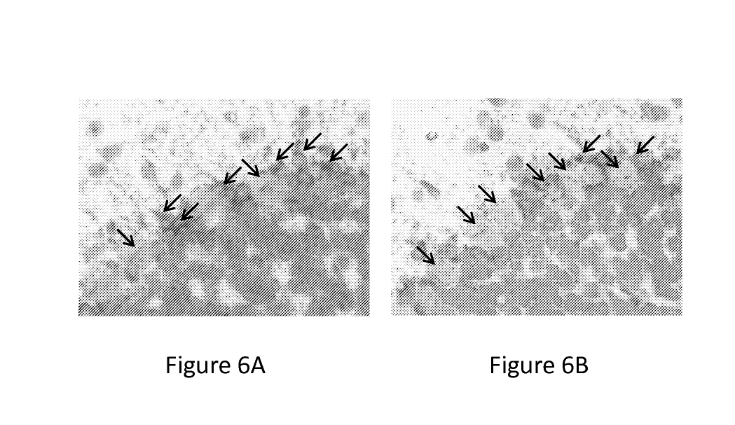

In an experiment demonstrating in situ detection of nucleic acid targets, Apoe (Apolipoprotein E) and Lgr5 (Leucine-rich repeat-containing G-protein coupled receptor 5) mRNA were simultaneously detected in Formalin-Fixed Paraffin-Embedded (FFPE) mouse cerebellum tissue using the methods described herein. First, two sets of paired target probes (TP1 and TP2) were hybridized to Apoe and Lgr5, respectively. Next, two different signal pre-amplifiers preAMP1 and preAMP2 were added to bind to TP1 and TP2, respectively, through cooperative hybridization. This was followed by adding two different sets of signal amplifier AMP1 and AMP2, which hybridize to preAMP1 and preAMP2, respectively. Two different sets of label probes, LP1 and LP2 were mixed and added to hybridize to AMP1 and AMP2, respectively. LP1 and LP2 were conjugated with alkaline phosphatase (AP) and horseradish peroxidase (HRP), respectively. After simultaneous hybridization of LP1 and LP2, substrates specific to AP and HRP were added sequentially to complete chromogenic reactions. The AP substrate used was FastRed, generating red colored precipitate. This was followed by reaction with HRP substrate DAB, to generate brown (FIG. 6A) precipitate, or a green HRP substrate, HRP-Green (42 Life Science), to produce green precipitate (FIG. 6B). This result demonstrates that the assay possesses excellent specificity because, as expected, the detected Apoe mRNA (red dots) were expressed only on astroglial cell body and their branches, while Lgr5 mRNA (Brown or green dots, detected by TP2 probe) was specifically located in large Purkinje neurons. This information on gene co-localization provides a useful tool to understand biological function at molecular level, can be used for drug discovery and development, and can be used for diagnostic purposes.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

1. A method of detecting two or more target nucleic acids, comprising:
(a) contacting a sample with two or more label probes, wherein each label probe comprises a distinct enzyme label and targets a distinct nucleic acid target;
(b) attaching the two or more label probes to a first target nucleic acid and a second target nucleic acid in the sample by hybridization;
(c) contacting the sample with a first substrate for the first enzyme of the first label probe;
(d) reacting the first substrate with the first enzyme, thereby producing a first detectable signal associated with the first target nucleic acid molecule;
(e) contacting the sample with a second substrate for the second enzyme of the second label probe;
(f) reacting the second substrate with the second enzyme, thereby producing a second detectable signal associated with the second target nucleic acid molecule; and
(g) detecting the first detectable signal and the second detectable signal, thereby detecting the first and second target nucleic acids in the sample.

2. The method of claim 1, wherein the detectable signal is chromogenic or fluorescent.

3. The method of claim 1, wherein the distinct enzyme labels are selected from horseradish peroxidase, alkaline phosphatase, β-galactosidase and glucose oxidase.

4. The method of claim 1, wherein the label probes are directly hybridized to the target nucleic acid molecules.

5. The method of claim 1, wherein the label probes are bound to the target nucleic acid molecules by a first and second intermediary complex.

6. The method of claim 5, wherein the intermediary complex comprises a molecule comprising a first region complementary to the target nucleic acid and a second region complementary to the label probe.

7. The method of claim 5, wherein the intermediary complex comprises an amplifier or preamplifier.

8. The method of claim 5, wherein the intermediary complex comprises an amplifier and preamplifier.

9. The method of claim 5, wherein a plurality of label probes are hybridized to one or more amplifiers.

10. The method of claim 5, wherein a plurality of amplifiers are hybridized to a preamplifier.

11. The method of claim 5, wherein the preamplifier is hybridized to the target nucleic acid.

12. The method of claim 5, wherein the intermediary complex comprises a target probe, amplifier or preamplifier.

13. The method of claim 5, wherein the intermediary complex comprises a target probe, amplifier and preamplifier.

14. The method of claim 12, wherein a plurality of label probes are hybridized to one or more amplifiers.

15. The method of claim 12, wherein a plurality of amplifiers are hybridized to a preamplifier.

16. The method of claim 12, wherein the preamplifier is hybridized to a target probe.

17. The method of claim 12, wherein the preamplifier is hybridized to two or more target probes.

18. A method of detecting two or more target nucleic acids, comprising:
(a) contacting a sample with two or more sets of label probes, wherein each set of label probes comprises a plurality of label probes, wherein the plurality of label probes within a set have the same enzyme label, and optionally wherein the plurality of label probes comprise label probes that bind to different regions of a target nucleic acid molecule; and wherein each set of label probes comprises a distinct enzyme label and targets a distinct nucleic acid target;
(b) attaching the two or more sets of label probes to a first target nucleic acid and a second target nucleic acid in the sample by hybridization;
(c) contacting the sample with a first substrate for the first enzyme of the first set of label probes;
(d) reacting the first substrate with the first enzyme, thereby producing a first detectable signal associated with the first target nucleic acid molecule;
(e) contacting the sample with a second substrate for the second enzyme of the second set of label probes;
(f) reacting the second substrate with the second enzyme, thereby producing a second detectable signal associated with the second target nucleic acid molecule; and
(g) detecting the first detectable signal and the second detectable signal, thereby detecting the first and second target nucleic acids in the sample.

19. The method of claim 18, wherein the detectable signal is chromogenic or fluorescent.

20. The method of claim 18, wherein the distinct enzyme labels are selected from horseradish peroxidase, alkaline phosphatase, β-galactosidase and glucose oxidase.

21-34. (canceled)
